(19) United States
(12) Patent Application Publication
(10) Pub. No.: US 2025/0134771 A1
(43) Pub. Date: May 1, 2025

(54) SECONDARY PACKAGING CONFIGURED TO HOLD AND/OR PREVENT INADVERTENT ACTUATION OF A DEVICE FOR ADMINISTERING A DRUG

(71) Applicant: CSP TECHNOLOGIES, INC., Auburn, AL (US)

(72) Inventors: Julien KLEIN, Niederbronn-les-Bains (FR); Ethan Ross PERDUE, Auburn, AL (US); Franklin Lee LUCAS, JR., Auburn, AL (US)

(21) Appl. No.: 19/011,017

(22) Filed: Jan. 6, 2025

Related U.S. Application Data
(63) Continuation of application No. PCT/US2023/069355, filed on Jun. 29, 2023.
(60) Provisional application No. 63/484,097, filed on Feb. 9, 2023.
(60) Provisional application No. 63/367,753, filed on Jul. 6, 2022.

(51) Int. Cl. A61J 1/14

(52) U.S. Cl. CPC A61J 1/1412; A61J 1/1468

(57) ABSTRACT

Secondary packaging configured to prevent inadvertent actuation of a device for administering a drug includes a body defining a cavity for at least partially surrounding the device for administering the drug. An active polymer component optionally can be located within an internal compartment of the secondary packaging. The active polymer component can include a mixture of 3 A molecular sieve, a base polymer, and optionally a channeling agent.

### CROSS-REFERENCE TO RELATED APPLICATIONS

The present application is a Continuation of International Application No. PCT/US2023/069355, titled “SECONDARY PACKAGING CONFIGURED TO HOLD AND/OR PREVENT INADVERTENT ACTUATION OF A DEVICE FOR ADMINISTERING A DRUG” and filed Jun. 29, 2023, which claims priority to U.S. Provisional Application No. 63/484,097, titled “SECONDARY PACKAGING CONFIGURED TO HOLD AND/OR PREVENT INADVERTENT ACTUATION OF A DEVICE FOR ADMINISTERING MEDICINE OR A DRUG” and filed on Feb. 9, 2023, and U.S. Provisional Application No. 63/367,753, titled “PACKAGING CONFIGURED TO PREVENT INADVERTENT ACTUATION OF A DEVICE DESIGN TO ADMINISTER MEDICINE OR A DRUG” and filed Jul. 6, 2022, which are each hereby incorporated by reference in their entirety.

### FIELD

The presently disclosed technology relates generally to packaging configured to hold or prevent inadvertent opening and/or actuation of a device designed to administer contents, such as but not limited to a drug. More particularly, in one optional embodiment, the presently disclosed technology relates to secondary packaging configured to hold or prevent inadvertent opening thereof and/or actuation of an inhaler therein.

### BACKGROUND

Devices for administering or releasing contents, such as drugs, are often stored or transported in secondary packaging. This secondary packaging protects the device(s), and can be discarded or reused once the device is removed from the secondary packaging for use by an end user or healthcare professional, for example.

The secondary packaging can include a desiccant plug in the interior thereof, as disclosed in U.S. Pat. No. 10,765,602. The desiccant can preserve or extend the useful life of the drugs within the device.

Secondary packaging can, from time to time and/or in a particular circumstance or use, inadvertently open. Inadvertent opening of the secondary packaging can unnecessarily or undesirably expose the device(s) therein to the external environment. Such exposure risks wasting or degrading the contents of the devices (e.g., in the event that the drug in a delivery device degrades in the presence of moisture), or damaging or misplacing the device(s). This is particularly problematic when the drug is either expensive or difficult to procure, or when the user may have difficulty quickly or reliably accessing replacement medicine needed to treat a dire medical situation.

### SUMMARY

In an effort to overcome the above-described and other drawbacks of the prior art, Applicant has investigated and identified various ways to prevent inadvertent opening of the secondary packaging.

Applicant has unexpectedly discovered that certain desiccant formulations used within secondary packaging can contribute to the inadvertent opening of the lid on the packaging. In particular, Applicant discovered that some desiccant formulations can create an elevated or heightened internal pressure within the closed secondary packaging. This increased pressure can cause or contribute to the inadvertent opening of the lid on the secondary packaging.

Accordingly, Applicant has unexpectedly discovered that using a particular type of desiccant formulation can reduce or help reduce pressure created within a closed secondary packaging, thereby preventing or at least reducing the likelihood of inadvertent opening of the lid on the secondary packaging.

It is an object of one optional embodiment of the presently disclosed technology to provide a cap and container assembly which provides a secure and sealed enclosure for a medication administration device therein, while also including components which prevent premature discharge of the medication and/or opening of the cap with respect to the container assembly.

Another object of one optional embodiment of the presently disclosed technology is to provide a medication delivery system that contains or houses a medication administration device in a manner that the medication administration device can be readily and quickly removed from a container for use.

### BRIEF DESCRIPTION OF THE DRAWINGS

The following detailed description of the presently disclosed technology, will be better understood when read in conjunction with the appended drawings, wherein like numerals designate like elements throughout. For the purpose of illustrating the presently disclosed technology, there are shown in the drawings various illustrative embodiments. It should be understood, however, that the presently disclosed technology is not limited to the precise arrangements and instrumentalities shown. In the drawings:

FIG. 1 is a perspective view of secondary packaging according to an optional aspect of the presently disclosed technology, wherein a cap is shown in a closed position;

FIG. 2 is a perspective view of the secondary packaging of FIG. 1, wherein the cap is shown in an open position and a portion of a device for administering contents therein is exposed;

FIG. 3 is a cross-sectional perspective view of the secondary packaging taken along line I-I of FIG. 1, wherein a portion of the device is shown in cross-section in a cavity of the secondary packaging;

FIG. 4 is a cross-sectional perspective view of the secondary packaging taken along line II-II of FIG. 1, wherein a portion of the device is shown in cross-section in the cavity of the secondary packaging;

FIG. 5 is a graph showing volume of gas released as a function of time during exposure at 50° C. according to the presently disclosed technology;

FIG. 6 is a graph showing the evolution of pressure inside a head space of secondary packaging of the presently disclosed technology as a function of time;

FIG. 7 is a perspective view of a secondary packaging according to an optional aspect of the presently disclosed technology, wherein a cap is shown in an open position;

FIG. 8 is a top plan view of the secondary packaging of FIG. 7;

FIG. 9A is a side elevation view of the secondary packaging of FIG. 7, wherein the cap is shown in a closed position;

FIG. 9B is magnified view of detail “A” of FIG. 9A;

FIG. 10 is a magnified view of a portion of the secondary packaging of FIG. 7;

FIG. 11 is a magnified view of a portion of the secondary packaging of FIG. 7; and

FIG. 12 is a magnified view of a portion of the secondary packaging of FIG. 7.

### DETAILED DESCRIPTION

While systems, devices and methods are described herein by way of examples and embodiments, those skilled in the art recognize that the presently disclosed technology is not limited to the embodiments or drawings described. Rather, the presently disclosed technology covers all modifications, equivalents and alternatives falling within the spirit and scope of the appended claims. Features of any one embodiment disclosed herein can be omitted or incorporated into another embodiment.

Any headings used herein are for organizational purposes only and are not meant to limit the scope of the description or the claims. As used herein, the word “may” is used in a permissive sense (i.e., meaning having the potential to) rather than the mandatory sense (i.e., meaning must). Unless specifically set forth herein, the terms “a,” “an” and “the” are not limited to one element but instead should be read as meaning “at least one.” The terminology includes the words noted above, derivatives thereof and words of similar import.

As used herein, “and/or” means that either or both of the items separated by such terminology are involved. For example, the phrase “A and/or B” would mean A alone, B alone, or both A and B.

As used herein, “generally” means “in a general manner” relevant to the term being modified as would be understood by one of ordinary skill in the art.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Generally speaking, as used herein, the term “moisture tight” is defined as having a moisture ingress (after three days) of less than 1500 μg of water, in another embodiment, less than 500 μg of water, in a further embodiment, less than 300 μg of water, in yet another embodiment, less than 150 μg of water, as determined by the following test method: (a) place one gram plus or minus 0.25 grams of molecular sieve in the container and record the weight; (b) fully close the container; (c) place the closed container in an environmental chamber at conditions of 80% relative humidity and 72° F.; (d) after one day, weigh the container containing the molecular sieve; (e) after four days, weigh the container containing the molecular sieve; and (f) subtract the first day sample from the fourth day sample to calculate the moisture ingress of the container in units of micrograms of water. A preferred rate of ingress of moisture into a moisture tight sealed container produced according to an aspect of the disclosed concept is in the range of about 200-300 μg/day of water or less. A “moisture tight” seal therefore is a sealing engagement that alone, or in combination with additional sealing engagements, help to render a container “moisture tight” per the above definition.

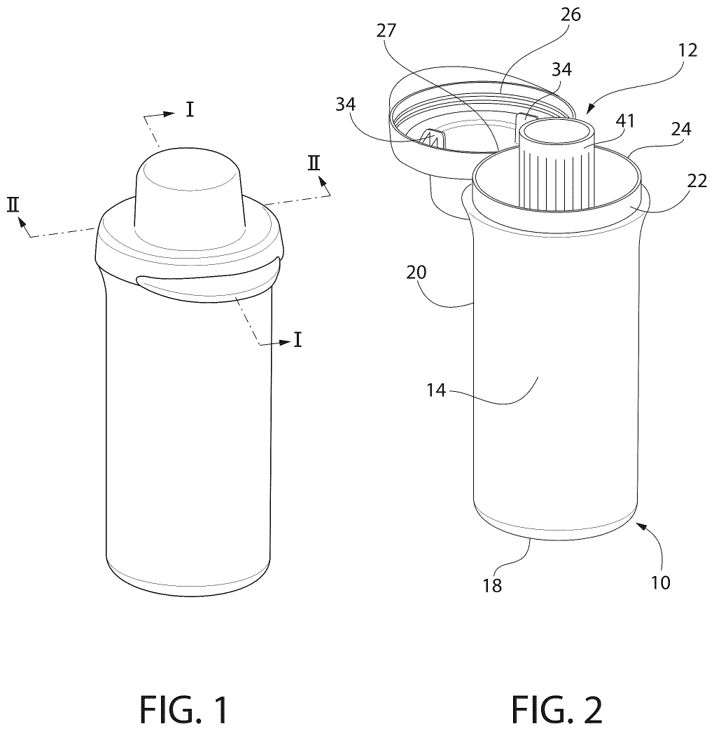

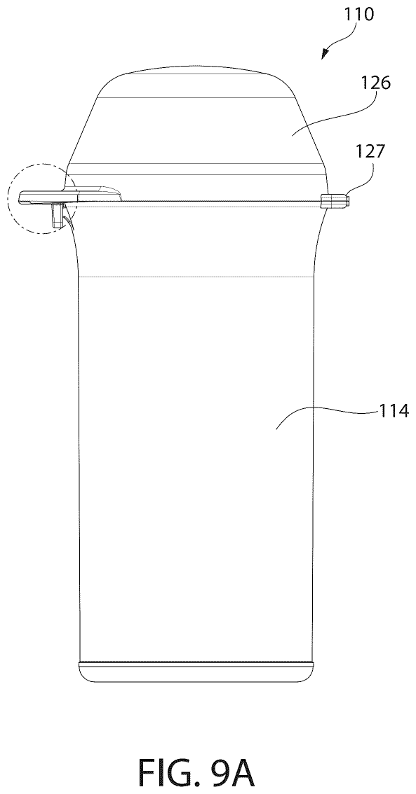

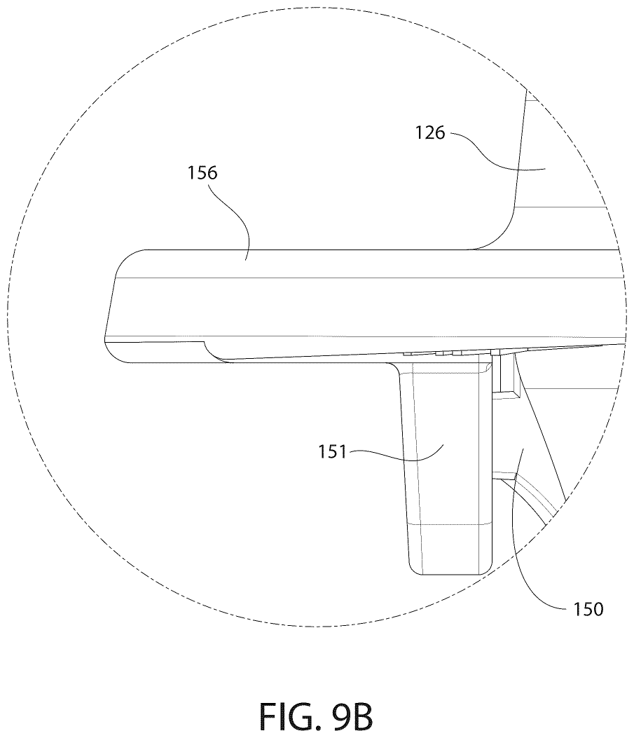

The term “moisture tight” is further discussed and/or defined in U.S. Pat. Nos. 11,192,698 and 11,325,771, and U.S. Patent Application Publication No. 2021/0008771, each of which are hereby incorporated by reference. For example, U.S. Pat. No. 11,192,698 (e.g., see FIGS. 1, 2, 7, and 9) discloses an elastomeric seal, optionally in the form of an annular ring, on an interior surface of a lid. This seal can be configured to engage at least a portion of the body when the cap is in the closed position to form a supplemental moisture tight seal between the cap and the body. Such a feature can optionally form at least one aspect of the presently disclosed technology.

As used herein, the term “resealable” means the lid of the container can be opened or reopened and closed or reclosed many times (e.g., more than 10 times) and still retain its moisture-tight properties.

As used herein, the term “secondary” in the phrase “secondary packaging” means that the packaging is intended not to directly contain or hold contents consumed by the user, but instead is intended to hold the packaging or drug delivery device that directly contains or holds the contents consumed by the user.

Referring now in detail to the various figures, wherein like reference numerals refer to like parts throughout, FIGS. 1-4 show a secondary packaging, generally designed 10, and at least one device, generally designated 12, such as a medication administration device. Each device 12 can be configured for storing, releasing, and/or administering contents therein, such as but not limited to a drug. The secondary packaging 10 can be configured to hold or container one or more of the devices 12 therein, and prevent inadvertent actuation of the device 12.

Optionally, the device 12 is an inhaler, such as but not limited to nasal drug delivery devices, nasal inhalers, metered-dose inhalers (MDIs), dry powder inhalers (DPIs), and soft mist inhalers (SMIs). The device 12 can be any from a wide range of medication administration devices and medications.

The device 12 can optionally include a spring to facilitate movement (e.g., expansion and contraction) of the device 12 from an activated position in which at least some of the contents of the device 12 are released or administered, to a non-activated position in which contents of the device 12 are prevented from exiting. However, the device 12 is not limited to being an inhaler, as the device 12 can be any structure configured to store, release, and/or administer contents held therein, such as an eyedropper or a pepper sprayer.

In one optional embodiment, the device 12 can employ a platform known as AMORPHOX™ as a carrier for nasal drug delivery, and is validated for small and large molecules. AMORPHOX™ can also serve as a carrier for Enzymes and Spike Proteins (including Sars-Cov2).

Optionally, the active pharmaceutical ingredient (API) stored within and/or delivered by the device 12 can be one or more of Naloxone, Ketamine, Midazolam, Fentanyl, Lorazepam, Glucagon, Nalmefene, Epinephrine, Apomorphine, Cetrorelix, Loxapine, Eletriptan, Ketorolac, and/or Olanzapine.

Optionally, the device 12 can be employed to store and/or deliver insulin and/or epinephrine and/or medicament to treat or reduce the symptoms of diabetes or asthma, for example.

The device 12 may be pre-filled and provide a medication in dosage form. In particular, the device 12 can be a dispersion device that delivers a medication in a dispersed form, such as in metered aerosol or spray devices. The device 12 can be pre-filled with the medication(s) in a finished dosage form. Embodiments include devices such as referenced in 68 Fed. Reg. 36,675, 36,676 and 36,680 (Jun. 18, 2003). Exemplary embodiments include intranasal aerosol and spray devices.

The secondary packaging 10 is particularly well suited for use with devices 10 that are used for administration of powder medications, such as intranasal delivery powders. The medications can be any that can be delivered by a dispersion device. The medication(s) can be associated with one or more other ingredients, as referenced, for example, at 21 C.F.R. § 314.3.

In an optional embodiment, the medication stored within the device 12 is a powder medicament, such as glucagon. The device 12 can be configured for the delivery of glucagon or another medicament in a simple, one-use nasal powder. This provides significant advantages over complicated systems that involve mixing liquid and powder together and then injecting the solution. The use of the device 12 by a patient has been determined to bring blood sugar levels close to normal within 30 minutes of taking the powdered glucagon.

In another optional embodiment, the medication is intranasal AMG504-1 product (from Locemia Solutions of Quebec Canada) containing 2 mg glucagon in 20 mg dry powder or 3 mg glucagon in 30 mg dry powder, depending on the dose. The nasal powder can be administered with a single-use, one-step dispensing device. The tip of the device can be inserted in one nostril, and the dose can be delivered by simply depressing an actuator 41 connected to a piston (not shown) that discharges the powder into the nostril. No cooperative measure is required from the patient, as absorption takes place through the nasal mucosa. The glucagon formulation is provided in the device 12, resulting in a highly effective protection for the viability of both the glucagon and its delivery device.

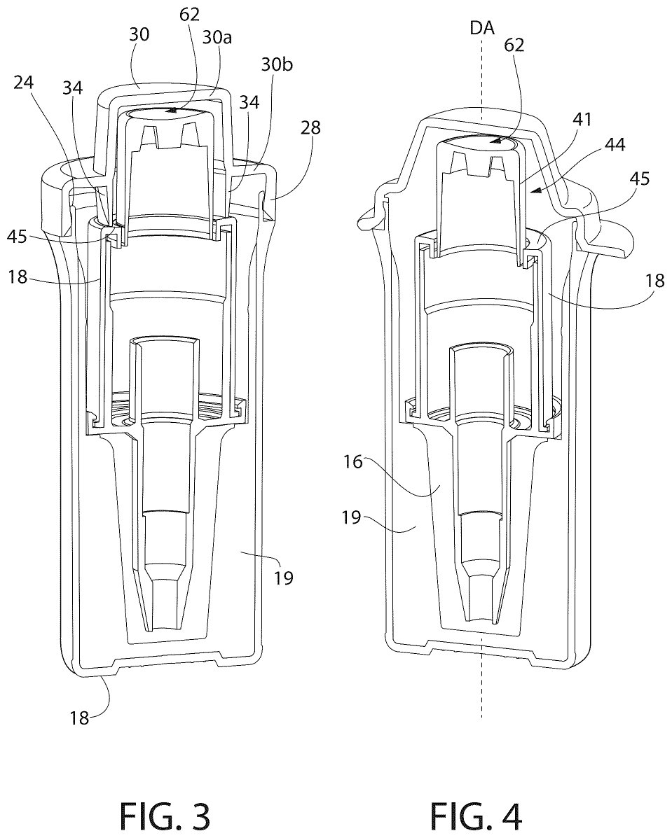

In one optional embodiment, as shown in FIGS. 3 and 4, the device 12 incudes a body 40 disposed generally longitudinally along a device axis DA, having a discharge end 42 and an opposed actuator end 44. The device 12 can include a medication reservoir 46 defined by body 40 and can contain a medication to be dispensed. The medication can be dispensed through a medication discharge port 48 which can include an opening communicating with the medication reservoir 46.

The device 12 can optionally include the actuator 41 at the actuator end 44 for ejecting the medication from the reservoir 46 through discharge port 46. The actuator 41 can optionally extend out of the actuator end 44 of the body 40. The actuator 41 can optionally be linearly movable from an extended position (not shown) to a depressed position (see FIGS. 3 and 4). The medication discharge port 48 can be sealed from communication with the medication reservoir 46 when actuator 41 is in the extended position to retain the contained medication in a sealed condition. The actuator 41 can be operable to discharge medication from the reservoir 46 upon axial movement from the extended position to the depressed position. During such movement, communication between the discharge port 48 and the reservoir 46 can be provided to allow for dispensing of the medication. Further details regarding the device 12 or similar devices that may be used according to optional aspects of the presently disclosed technology are disclosed in U.S. Pat. No. 10,894,133, which is incorporated by reference herein in its entirety.

The secondary packaging 10 can include a body or container 14 defining a cavity 16 for at least partially surrounding the device 12. The body 14 of the secondary packaging 10 can include a base 18 and a sidewall 20 extending upwardly therefrom to form the cavity 16. An end of the sidewall 20 opposite the base 18 can include a rim 22 that surrounds and/or defines an opening 24 leading to the cavity 16. Optionally, the body 14 can be cylindrical.

In the present optional embodiment, the body 14 of the secondary packaging 10 is in the form of a cylinder or bottle and the sidewall 20 can be round and unitary. However, bodies or containers according to the disclosed concept may be other shapes, e.g., rectangular cuboid, and thus have more than a single continuous (e.g., round) sidewall.

As shown in FIGS. 3 and 4, at least a portion of the cavity 16 can be spaced inwardly from an interior surface of the sidewall 20 and/or upwardly from an interior surface of the base 18. This spacing can be the result of an object 19 placed or located within the body 14. The object 19 can be fixed with respect to the body 14. Optionally, the object 19 can be an active member, such as but not limited to desiccant or an oxygen scavenger. The size, shape, configuration and/or location of the object 19 can contribute to preventing inadvertent actuation of the device 12.

Optionally, the object 19 can be formed as part of a two-shot injection molding process or over-molding process with the body 14. Optionally, the object 19 can be formed as a separate plug from the body 14, which is inserted into and optionally interlocks with mating geometry or optionally is press-fit into the body 14, for example.

As shown in FIGS. 1-4, the secondary packaging 10 can include a cover or cap 26. Optionally, the cap 26 is rigid. In the present optional embodiment, the cap 26 is movable with respect to the body 14 between a closed position (e.g., FIGS. 1, 2, and 4) in which the cap 26 and the body 14 can optionally form a seal, such as but not limited to a moisture-tight seal and/or a hermetic seal, and an open position (e.g., FIG. 3) in which the opening 24 of the body 14 is exposed. In one embodiment, the cap 26 can include an active member.

Referring to FIGS. 1 and 3, the cap 26 can include an annular skirt 28 extending downwardly (directly or indirectly) from a periphery of a top portion or base 30 of the cap 26. In the present optional embodiment, the base 30 is at least partially planar. Alternatively, the base 30 can be partially or completely arcuate or flat. Optionally, as shown in FIGS. 1, 3, and 4, the base 30 can include an upper planar surface 30a spaced-apart from a lower planar surface 30b, which can be connected by a vertical or slanted surface. Thus, in one optional embodiment, the base 30 can form a cup shaped segment. The skirt 28 can include a first end proximate the base 30 and an opposing second free end. The cap 26 is optionally made primarily from one or more injection moldable thermoplastic materials, including, for example, a polyolefin such as polypropylene or polyethylene.

Optionally, a hinge 27 can connect the cap 26 to the body 14. The hinge 27 may be in the form of a living hinge, such as including a continuous material bridge connecting the cap 26 to the body 14. At least a portion of hinge 27 may be flat, instead of matching the radius of curvature of the body 14.

In an alternative embodiment, the hinge 27 can be an axle-like member extending horizontally along the outside of the body 14. The axle can be held at a position spaced from the exterior of the outside of the body 14, such as by a pair of outwardly extending flanges. In this embodiment, the cap 26 can include a partially cylindrical member that is received over and pivots around the axle. Alternatively, these structures may be reversed with the cap 26 including the pivot axle and the body 14 including the cylindrical component. In these alternate embodiments, the cap 26 and the body 14 can be formed separately and assembled together.

The cap 26 can optionally be resealable with respect to the body 14, whether or not the secondary packaging 10 includes a hinge 27 or if the cap 26 and the body 14 are fully detachable.

Optionally, when the device 12 is within the cavity 16, at least a portion of the device 12 can be configured to prevent and/or includes means for preventing movement of the device 12 that would otherwise release or administer the drug.

More particularly, in one optional embodiment as shown in FIGS. 3 and 4, at least one or two or more spaced-apart projections 34 extend from an inner surface of the cap 26. Each projection 34 can be positioned and/or configured to bear against a non-actuator portion of the device 12. The one or more projections 34 can retain the device 12 in the cavity 16 without pressure being applied to the actuator 44 while the cap 26 is in the closed position. In particular, each projection 34 can extend from the cap 16 toward bottom wall 18 when the cap 16 is in the closed position. In one example, each projection 34 is formed integrally from the same material with the cap 26 using a molding process. In other examples, each projection 34 can be formed separately and bonded or otherwise securely fixed to the cap 26.

Each projection 34 can optionally be made from a material that provides a rigid structure. In one example, the projections 34 are configured to maintain their relative orientation and positioning relative to the cap 26 (and each other if more than one) and the non-actuator portion of body 40 of the device 12 to avoid movement of the ends of the projections 34 to an undesirable position that could cause premature actuation of the device 12. A rigid projection 34 can maintain its relative axial position and radial position relative to the cap 26 and the device 12.

The device 12 is shown received within the body 14 of the secondary packaging 10 in FIGS. 2-4. At least a portion of the actuator 44 can extend upwardly beyond a plane defined by the opening 24 and which extends parallel to the bottom wall 18 when the device 12 is properly and/or fully inserted into the body 14.

Optionally, as shown in FIGS. 3 and 4, the body 18 of the device 12 can include a circumferential surface or wall 45 surrounding, and coaxial with, the actuator 44 about a device axis DA and facing in the direction away from the bottom wall 18 when the device 12 is in the secondary packaging 10. Each projection 34 can be axially extending parallel to the device axis DA when the cap 26 is in the closed position. Optionally, each projection 34 can have end contact surfaces positioned to bear against circumferential surface 45, representing the non-actuator portion, which is radially outside and clear of the actuator 44, in the event that the device 12 is urged to move axially in a direction away from bottom wall 18. With a rigid projection 34, the end contact surface maintains its relative axial position and radial position relative to the cap 26 and the circumferential surface 45 of the device 12. Each projection 34 may include a support rib along a radially outer surface thereof. Other portions of the device 12 may be used as the non-actuator portion that is contacted by the projection(s) 34.

Optionally, each projection 34 is provided to prevent the device 12 from moving away from bottom wall 18 a distance that would cause the actuator 44 to bear against an interior axial surface of the cap 26. If such contact was allowed, it would be possible for the device 12 to be damaged or to prematurely discharge the medication.

Each projection 34 is positioned and configured to interfere with movement of the device 12 within the secondary packaging 10 to allow at most a predetermined amount of movement of the device 12 in the direction away from bottom wall 18 and toward cap 26. In some embodiments, there may be constant contact between each projection 34 and the circumferential surface 45 when the cap 26 is in the closed position (see FIG. 3). However, in some embodiments, there may not be constant contact between each projection 34 and the circumferential surface 45. For example, in one optional embodiment, a first gap or space (not shown) can be provided between the end contact surface of each projection 34 and the circumferential surface 45. The first gap can optionally be between 0.1 mm and 1.865 mm. If an external force is applied that would cause the device 12 to move away from bottom wall 18, then the contact surface of each projection 34 can serve as an end stop to prevent or limit such movement.

Optionally, as shown in FIGS. 3 and 4, a second cap G2 can be located between an end surface of the actuator 44 and an interior surface of the base 30 of the cap 26. The second gap G2 can be at least slightly larger than the first gap G1, which prevents the actuator 44 from contacting the interior surface of the base 30.

In one optional embodiment, two spaced-apart projections 34 are disposed circumferentially away from one another, and the hinge 27 is disposed circumferentially between the two projections 34.

Optionally, a plug or a lip seal can depend downwardly and/or inwardly from the base 30, such as the upper planar surface 30a or the lower planar surface 30b of the cap 26, such as described in Applicant's U.S. Pat. No. 11,352,177, which is hereby incorporated by reference in its entirety.

For certain uses, a child-resistant cap may be desired, but may not be necessary for all applications. Thus, child-resistant and non-child-resistant caps are contemplated. If a child-resistant feature is provided, the child-resistant feature optionally requires that force in more than one single direction is applied to the cap to remove the cap from the body. For example, the cap may require a user to press downward (first direction) before rotating the cap (second direction) in order to remove the cap from the body. Alternative child-resistant features, if desired, are also contemplated.

In one optional embodiment, at least one or both of the body 14 and the cap 26 are formed of injection molding, such as a two-shot injection molding process. Applicant unexpectedly discovered that this also helps to prevent the problem of pre-mature actuation of the device 12.

Optionally, the secondary packaging 10, for example the cap 26 and/or the body 14 thereof, can include the object 19, an active member, or an active polymer component that is affixed to a portion thereof or integral therewith. For example, the active polymer component can be part of the molding process of the body 14 and/or the cap 26, such that the end user cannot visually identify or physically/manually detach the active polymer component from the body 14 and/or the cap 26. Optionally, the body 14 and/or the cap 26 can be formed of a two-shot or three-shot injection molding process, as described in detail below.

The active polymer component can include a base polymer entrained with one or more active agents (e.g., a mineral desiccant) and thus may be referred to herein as a polymer entrained with an active agent or entrained polymer. The active agent in the active polymer component may include an absorbing material, a releasing material and/or an activation material. Optionally, the active polymer component is a three-phase desiccant entrained polymer. The active polymer component can be provided in different shapes, volumes and/or configurations. In one exemplary embodiment, the active polymer component is in the form of a solid plug or generally planar member, extending into an interior space of the cap 26.

In one embodiment, active polymer component is a desiccant entrained polymer that is a unitary component made of a single piece of material. An entrained polymer, whether entrained with desiccant or another active agent, may include a base polymer (for structure), a desiccant (or other active agent) and optionally a channeling agent. These types of active entrained polymers and methods of making and using the same are disclosed, e.g., in Applicant's U.S. Pat. Nos. 5,911,937, 6,214,255, 6,130,263, 6,080,350, 6,174,952, 6,124,006 and 6,221,446, and U.S. Pat. Pub. No. 2016/0039955. Optionally, the entrained polymer may be in the form of a film that is loose or optionally heat staked to a surface.

Alternatively, the desiccant may include loose desiccant beads or a sachet containing the same. While the exemplary embodiments herein reflect active polymer component being attached, for example, to the cap 26, it is contemplated that an active agent can be located at other locations and/or positions, such as on a sidewall of the body.

In the embodiment where each active member contains a desiccant, moisture absorption is desired. However, where moisture absorption is not desired, the active member can include alternative active agents. For example, in another embodiment, the active member contains a material selected from the group consisting of activated carbon, carbon black, ketjenblack, and diamond powder. In a further embodiment, an active agent including one or more layers of the active member contains a material such as absorption microspheres, BaTiO3, SrTiO3, SiO2, Al2O3, ZnO, TiO2, MnO, CuO, Sb2O3, silica, calcium oxide and ion exchange resins. In yet another embodiment, the absorbing agent containing layer of the active member contains two or more types of absorbing agents. The suitable absorbing agent is chosen to achieve absorption of the desired vapor or gas for the desired end use (e.g., absorption of moisture, oxygen, carbon dioxide, nitrogen or other undesired gases or vapors).

The active member (whether desiccant, oxygen scavenger, a releasing material or ingredient, etc., or combination thereof) is capable of acting on, interacting or reacting with a selected material (e.g., moisture or oxygen). Examples of such actions or interactions may include absorption, adsorption (sorption, generally) or release of the selected material. Each active member can be extruded or molded, for example. Optionally, the active member can be formed in a desired shape or pattern (e.g., on a backing) via an in-line melt adhesion thermal bonding process.

The active member can include an “active ingredient” in a base material. The active ingredient(s) (i) can be immiscible with the base material (e.g., polymer) and when mixed and heated with the base polymer and a channeling agent, will not melt, i.e., has a melting point that is higher than the melting point for either the base polymer or the channeling agent, and/or (ii) acts on, interacts or reacts with a selected material. The term “active ingredient” may include but is not limited to materials that absorb, adsorb or release the selected material(s). Active ingredients, according to the presently disclosed technology, may be in the form of particles such as minerals (e.g., molecular sieve or silica gel, in the case of desiccants), but the presently disclosed technology should not be viewed as limited only to particulate active agents. For example, in some embodiments, an oxygen scavenging formulation may be made from a resin which acts as, or as a component of, the active agent.

As used herein, the term “base material” is a component (preferably a polymer) of an entrained active material, other than the active agent, that provides structure for the entrained material.

As used herein, the term “base polymer” is a polymer optionally having a gas transmission rate of a selected material that is substantially lower than, lower than or substantially equivalent to, that of the channeling agent. By way of example, such a transmission rate would be a water vapor transmission rate in embodiments where the selected material is moisture and the active ingredient is a water absorbing desiccant. The primary function of the base polymer is to provide structure for the entrained polymer. Suitable base polymers may include thermoplastic polymers, e.g., polyolefins such as polypropylene and polyethylene, polyisoprene, polybutadiene, polybutene, polysiloxane, polycarbonates, polyamides, ethylene-vinyl acetate copolymers, ethylene-methacrylate copolymer, poly (vinyl chloride), polystyrene, polyesters, polyanhydrides, polyacrylonitrile, polysulfones, polyacrylic ester, acrylic, polyurethane and polyacetal, or copolymers or mixtures thereof.

Referring to such a comparison of the base polymer and channeling agent water vapor transmission rate, in one embodiment, the channeling agent has a water vapor transmission rate of at least two times that of the base polymer. In another embodiment, the channeling agent has a water vapor transmission rate of at least five times that of the base polymer. In another embodiment, the channeling agent has a water vapor transmission rate of at least ten times that of the base polymer. In still another embodiment, the channeling agent has a water vapor transmission rate of at least twenty times that of the base polymer. In still another embodiment, the channeling agent has a water vapor transmission rate of at least fifty times that of the base polymer. In still another embodiment, the channeling agent has a water vapor transmission rate of at least one hundred times that of the base polymer.

As used herein, the term “channeling agent” or “channeling agents” is defined as a material that is immiscible with the base polymer and has an affinity to transport a gas phase substance at a faster rate than the base polymer. Optionally, a channeling agent is capable of forming channels through the entrained polymer when formed by mixing the channeling agent with the base polymer. Optionally, such channels are capable of transmitting a selected material through the entrained polymer at a faster rate than in solely the base polymer.

As used herein, the term “channels” or “interconnecting channels” is defined as passages formed of the channeling agent that penetrate through the base polymer and may be interconnected with each other.

As used herein, the term “entrained polymer” is defined as a monolithic material formed of at least a base polymer with an active agent and optionally also a channeling agent entrained or distributed throughout. An entrained polymer thus includes two-phase polymers and three phase polymers. A “mineral loaded polymer” is a type of entrained polymer, wherein the active agent is in the form of minerals, e.g., mineral particles such as molecular sieve or silica gel. The term “entrained material” is used herein to connote a monolithic material comprising an active agent entrained in a base material wherein the base material may or may not be polymeric.

As used herein, the term “monolithic,” “monolithic structure” or “monolithic composition” is defined as a composition or material that does not consist of two or more discrete macroscopic layers or portions. Accordingly, a “monolithic composition” does not include a multi-layer composite.

As used herein, the term “phase” is defined as a portion or component of a monolithic structure or composition that is uniformly distributed throughout, to give the structure or composition it's monolithic characteristics.

As used herein, the term “selected material” is defined as a material that is acted upon, by, or interacts or reacts with an active agent and is capable of being transmitted through the channels of an entrained polymer. For example, in embodiments in which a desiccant is used as an active agent, the selected material may be moisture or a gas that can be absorbed by the desiccant. In embodiments in which a releasing material is used as an active agent, the selected material may be an agent released by the releasing material, such as moisture, fragrance, or an antimicrobial agent (e.g., chlorine dioxide). In embodiments in which an adsorbing material is used as an active ingredient, the selected material may be certain volatile organic compounds and the adsorbing material may be activated carbon.

As used herein, the term “three phase” is defined as a monolithic composition or structure including three or more phases. An example of a three phase composition according to the presently disclosed technology would be an entrained polymer formed of a base polymer, active agent, and channeling agent. Optionally, a three phase composition or structure may include an additional phase, e.g., a colorant.

Entrained polymers may be two phase formulations (i.e., comprising a base polymer and active ingredient, without a channeling agent) or three phase formulations (i.e., comprising a base polymer, active agent and channeling agent). Entrained polymers are described, for example, in U.S. Pat. Nos. 5,911,937, 6,080,350, 6,124,006, 6,130,263, 6,194,079, 6,214,255, 6,486,231, 7,005,459, and U.S. Pat. Pub. No. 2016/0039955.

An entrained material or polymer includes a base material (e.g., polymer) for providing structure, optionally a channeling agent and an active agent. The channeling agent forms microscopic interconnecting channels through the entrained polymer. At least some of the active ingredient is contained within these channels, such that the channels communicate between the active ingredient and the exterior of the entrained polymer via microscopic channel openings formed at outer surfaces of the entrained polymer. The active ingredient can be, for example, any one of a variety of absorbing, adsorbing or releasing materials, as described in further detail below. While a channeling ingredient is preferred, the presently disclosed technology broadly includes entrained materials that optionally do not include channeling agents, e.g., two phase polymers.

In any embodiment, suitable channeling agents may include a polyglycol such as polyethylene glycol (PEG), ethylene-vinyl alcohol (EVOH), polyvinyl alcohol (PVOH), glycerin polyamine, polyurethane and polycarboxylic acid including polyacrylic acid or polymethacrylic acid. Alternatively, the channeling agent can be, for example, a water insoluble polymer, such as a propylene oxide polymerisate-monobutyl ether, such as Polyglykol B01/240, produced by CLARIANT. In other embodiments, the channeling agent could be a propylene oxide polymerisate monobutyl ether, such as Polyglykol B01/20, produced by CLARIANT, propylene oxide polymerisate, such as Polyglykol D01/240, produced by CLARIANT, ethylene vinyl acetate, nylon 6, nylon 66, or any combination of the foregoing.

Suitable active ingredients according to the presently disclosed technology include absorbing materials, such as desiccating compounds. If the active agent is a desiccant, any suitable desiccant for a given application may be used. Typically, physical absorption desiccants are preferred for many applications. These may include molecular sieves, silica gels, clays and starches. Alternatively, the desiccant may be a chemical compound that forms crystals containing water or compounds which react with water to form new compounds.

Optionally, in any embodiment, the active agent may be an oxygen scavenger, e.g., an oxygen scavenging resin formulation.

In one optional embodiment, the active agent is or includes 3 A molecular sieve. The pore diameter or a molecular sieve is often measured in angstroms (A), and 3 A molecular sieve refers to the pore size of 3 angstroms. Applicant unexpectedly discovered that use of 3 A molecular sieve, especially as compared to and/or in place of 4 A molecular sieve, helps to reduce pressure build-up during required testing of the secondary packaging 10. By reducing pressure build-up, it is less likely that the secondary packaging 10 will inadvertently open, thereby risking contamination and/or premature activation of the device 12. Additional discussion of the benefits of 3 A molecular sieve are discussed in Applicant's WO 2022/032278, which is hereby incorporated by reference.

Optionally, the mass of the object 19 is exactly or approximately 14.6 grams, which includes exactly or approximately 9.2 grams of 3 A molecular sieve. Optionally, the mass of the object 19 can be from 13-16 grams. Optionally, the mass of the 3 A molecular sieve can be from 8-10 grams.

Applicant believes that the smaller angstrom size of 3 A molecular sieve, as compared to 4 A molecular sieve, reduces off-gassing, which thereby produces less off gassing pressure and/or reduces the pressure within the secondary packaging 10. This is particularly true during the testing that requires extended time periods (e.g., 96 hours) at which the secondary packaging 10 must be maintained in a high temperature (e.g., 50° C.) or low temperature environment. Prior to this unexpected discovery, 3 A molecular sieve was used in instances where additional absorption capacity was desired.

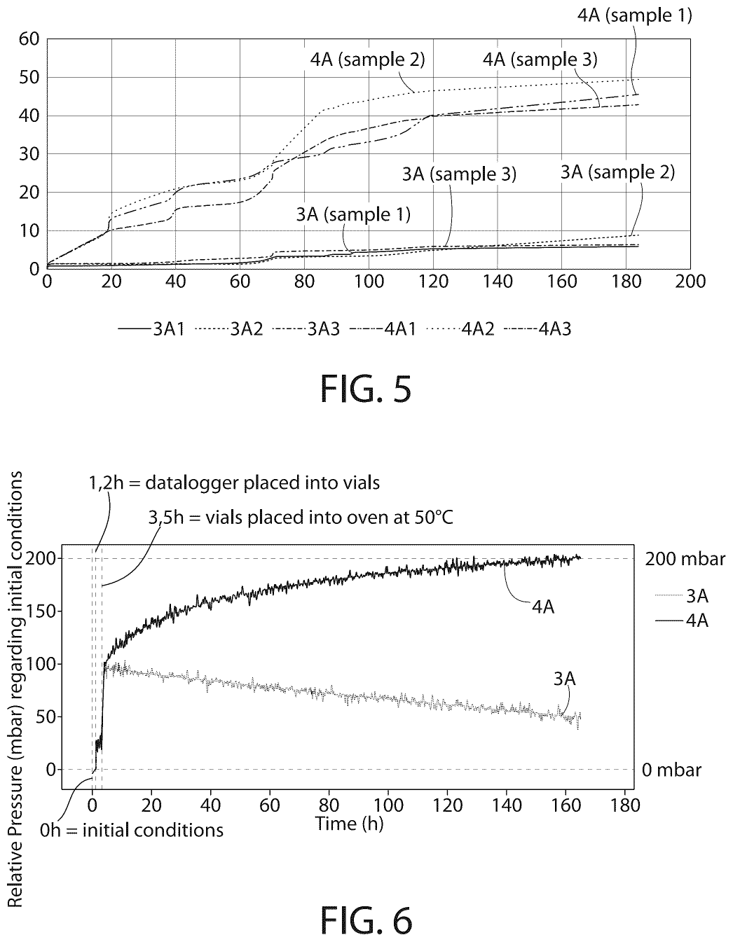

FIG. 5 shows the results of comparative testing between 3 A molecular sieve desiccant and 4 A molecular sieve desiccant, where the volume of gas released is charted with respect to time. The testing was conducted by exposing various samples of 3 A and 4 A molecular sieve to 50° C. temperature for 184 hours. Each 14 gram 3 A and 4 A molecular sieve sample was placed in a tight tube, each of which were connected to a measuring cylinder placed in water. During the experiment, all of the gas released by the samples placed in the tight tubes was directly stored in the measuring cylinders. In this manner, the volume of gas released could be observed.

As shown in FIG. 5, after 184 hours of exposure at 50° C., it was observed that the 3 A based formulation sample (14 gr of compound) released about 7.2 mL of gas. Regarding the 4 A based formulation, the same quantity of compound released 46 mL of gas, which is about 7 times more than the 3 A based formulation. These results are consistent with the pressure monitoring test performed, as discussed immediately below.

FIG. 6 shows the result of a pressure monitor testing of the secondary packaging 10 of the presently disclosed technology. The object of this testing was to determine the over pressure difference inside the head space of the secondary packaging 10 employing 3 A molecular sieve desiccant versus packaging employing 4 A molecular sieve desiccant when both are exposed to 50° C. temperature for 7 days.

One STAR ODDI™ data logger was used for each of the two secondary packaging (i.e., vial). Each vial was exposed to 50° C. temperature (uncontrolled RH) for 7 days. After 7 days, data from the data loggers was extracted and analyzed. The pressure was measured every 15 minutes for the 7 day period.

It was observed that when both 3 A and 4 A based desiccant vials were exposed to a temperature of 50° C., pressure inside the vials initially increased due to gas expansion. This increase in pressure lasted for a few hours until the pressure inside the vials reached approximately 100 mBars. During this phase, no difference between the two desiccant formulations was observed.

After approximately 5 hours of exposure at 50° C., however, a significant difference between the vials using 3 A molecular sieve and 4 A molecular sieve was observed. In particular, the pressure increased up to approximately 200 mBars after 7 days inside the vials made of or using 4 A molecular sieve, while the pressure inside the vials made of or using 3 A molecular sieve decreased from the peak of 100 mBars to approximately 50 mBars during this period.

It was determined that the 4 A molecular sieve released a significant amount of gas inside the head space of the vial when exposed to a temperature of 50° C. This led to an over pressure of 200 mBars inside the vials given the tightness of the vials. In contrast, it was determined that the 3 A molecular sieve did not release a significant quantity of gas and the over pressure inside the vials after 7 days was very low. These findings are significant in at least two respects. First, the final pressure measurements under the stated conditions showed that the packaging with 3 A molecular sieve had a substantially lower internal pressure than the packaging with an equivalent mass of 4 A molecular sieve. Second, not only did the 3 A molecular sieve result in a substantially lower end pressure measurement, but after initially peaking, the packaging with the 3 A molecular sieve experienced a pressure drop while the packaging with the 4 A molecular sieve continued to increase in pressure. These findings are noteworthy. In practical terms, the significantly lower pressure inside the packaging using 3 A molecular sieve has a substantially lower probability of inadvertently opening than the packaging using an equivalent mass of 4 A molecular sieve.

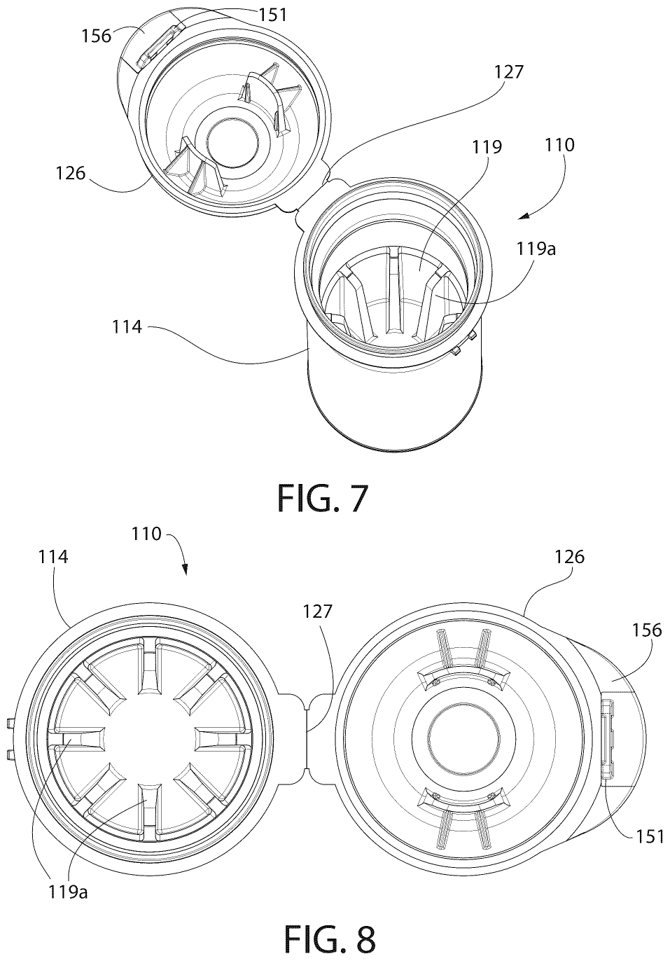

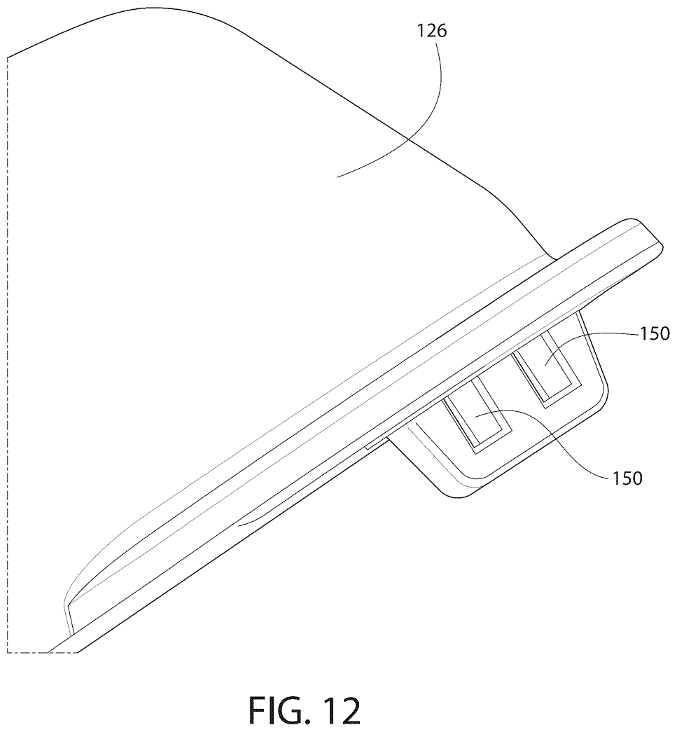

FIG. 7-12 show another embodiment of the presently disclosed technology. Similar or identical structure as between the embodiment of FIGS. 7-12 and the embodiment of FIGS. 1-4 is distinguished in FIGS. 7-12 by a reference number with a magnitude one hundred (100) greater than that of FIGS. 1-4. Description of certain similarities between the embodiment of FIGS. 1-4 and the embodiment of FIGS. 7-12 may be omitted herein for convenience and brevity only. Features of one embodiment may be omitted or added to the other embodiment.

Optionally, the maximum height of the secondary packaging 110 is 81.17 mm, or optionally from 79 mm to 83 mm. Optionally, the maximum diameter of the body 114 of the secondary packaging 110 is 34.98 mm, or optionally from 33 mm to 36 mm. Optionally, the maximum width (i.e., cap 126 plus hinge 127) of the secondary packaging 110 is 43.97 mm, or optionally from 42 mm to 45 mm. Optionally, the internal volume of the secondary packaging is 36,651 mm³, optionally from 30,000 to 40,000 mm³, optionally from 34,000 to 38,000 mm³.

As shown in FIGS. 7-9A, the cap 126 can optionally be formed of a semi-rigid material with the hinge 127 directly molded to the body 114 of the secondary packaging 110. One or more of these components can optionally be formed from polypropylene.

Referring to FIGS. 7 and 8, the object 119 can optionally include a plurality of circumferentially spaced-apart fins or ribs 119a each extending inwardly from the body 114. Each fin 119a can be tapered, such that a width thereof, as measured radially from a geometric center of the body 114 toward the outer wall thereof, is smaller at or near a top of the fin 119a than at or near a bottom of the fin 119a. Such a shape or configured can facilitate insertion of the device (not shown) into the secondary packaging 110 and/or facilitate preventing inadvertent actuation of the device. Further, such a shape increases the surface area of the object 119, which can be beneficial where the object is an active polymer. In this way, the object 119 has more exposed surface area that may be used to provide the desired activity, e.g., moisture adsorption. In the same or a different embodiment, a thickness of each fin 119a, as measured perpendicularly to the width, can be the same throughout a height of the fin 119a. Alternatively or additionally, the object 119 can form a cylinder that receives at least a portion of the device therein.

As shown in FIGS. 7-9B, the cap 126 can optionally include a tab 156 extending laterally outwardly therefrom. The tab 156 can be sized, shaped, and/or configured to be engaged by a user, such as by a thumb of a user, to help move the cap 126 between the open and closed position. The tab 156 can optionally be positioned diametrically opposed to the hinge 127.

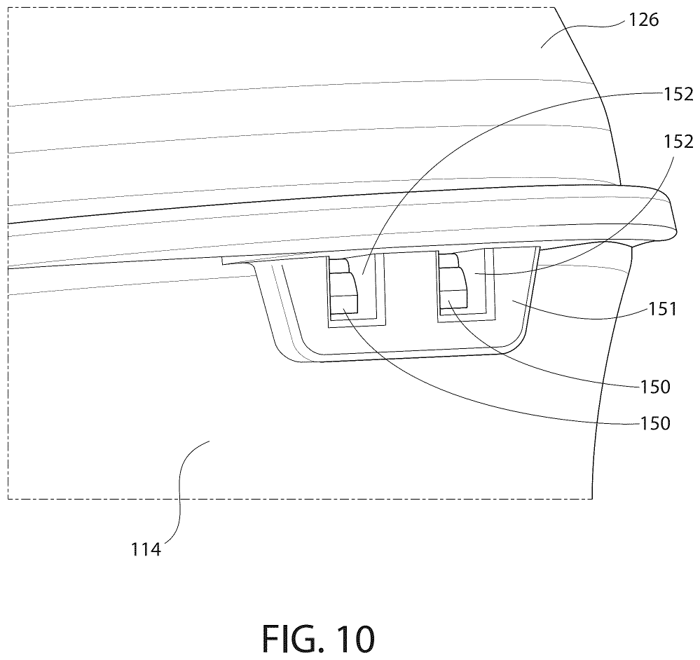

Referring to FIGS. 10-12, the cap 126 and/or the body 114 can be formed with a locking feature. The locking feature can be configured to hold the cap 126 in the closed position with respect to the body in the absence of an intended separation or relative movement of the two components, even with the presence of an internal pressure within the cavity of the body 114. Any such locking mechanism can be used that provides sufficient security to hold the components in a locked condition, while also permitting a reasonable release of the locking mechanism when desired.

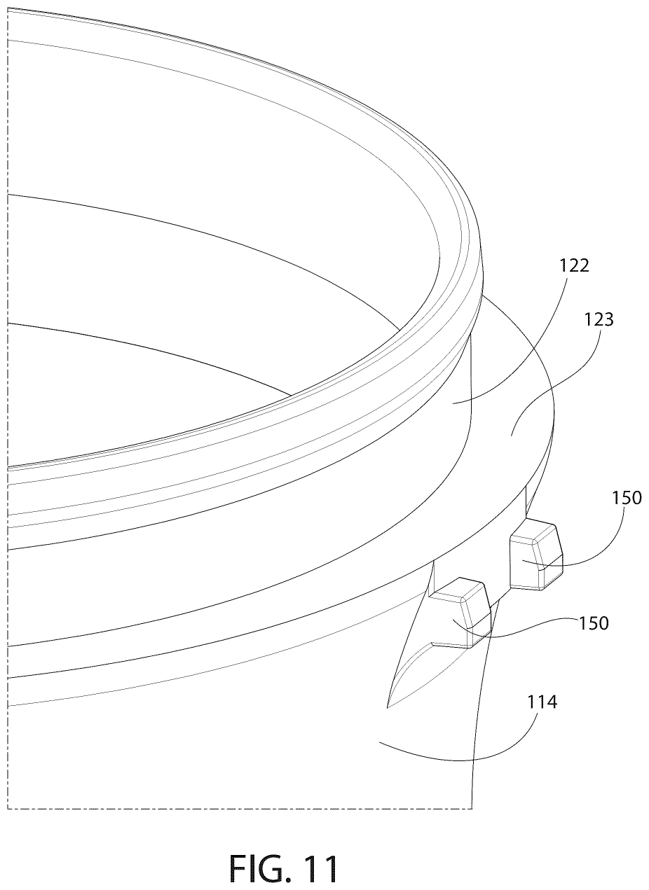

Optionally, the locking feature can include at least one rib 150 designed to prevent unintentional opening and/or pre-opening of the secondary packaging 110. A center of the locking feature can be located opposite the hinge 127, i.e., approximately 180° from the center of the hinge 127. Such configuration would help to maximize the closure effect of the locking feature and help ensure consistent opening and closing force of the secondary packaging 110. As shown in FIG. 11, each rib 150 can be located beneath the rim 122 and/or a lip 123 of the body 114.

In an optional embodiment, two spaced-apart ribs 150 can extend radially outwardly from the sidewall of the body 114, and the cap 126 can include two corresponding spaced-apart openings 152 in a latch 151 attached to and depending from an underneath surface of the tab 156 of the cap 126. Each opening 152 can be sized, shaped, and/or configured to receive at least a portion of one of the ribs 150 when the cap 126 is in a closed position (see FIG. 10).

In operation of one optional embodiment, a user can apply an axial force (e.g., upward force) to an underside of the tab 156 to cause the tab 156 to pivot (e.g., upwardly) at least slightly due to the flexibility of the tab 156, thereby moving the latch 151 in a manner (e.g., outwardly) such that each opening 152 is removed and/or separated from the respective rib 150 and the latch 151 clears the respective rib 150 to permit the cap 126 to move to from the closed to the open position.

In one optional embodiment, to disengage the locking feature, no actions other than retaining the body 114 (e.g., with one's hand) and applying upward force to the underside of the tab 156 are required.

As a slight variation, there may be a single rib or more than two ribs with one or more corresponding openings. Additional alternatives for locking features according to optional aspects of the disclosed concept are any version of the attachment mechanism disclosed in Applicant's U.S. Pat. No. 11,352,177, which is disclosed above.

Optionally, in any embodiment, the locking feature may be implemented in combination with the annular skirt 28 extending downwardly from a periphery of a base of the cap 26, 126. In such an embodiment the annular skirt or a portion thereof can serve as the depending latch described above.

Optionally, the presently disclosed technology can include a rim of the body having an outer surface that includes an undercut relative to a central axis of the body. The undercut can have a surface that mates with a corresponding surface of the skirt in a snap fit closure configuration when the cap is closed. The snap fit closure configuration can inhibit inadvertent opening of the cap and can form a moisture-tight seal between the body and the cap. One example of such an arrangement is shown and described in Applicant's U.S. Pat. No. 11,192,698, which is hereby incorporated by reference in its entirety.

Optionally, a snap fit closure and a moisture-tight seal between the body and cap can be combined with the locking feature to further ensure against inadvertent opening of the cap with respect to the body, due at least in part, e.g., to pressure buildup within the package. Such a combination of the snap fit and seal/closure can be between the skirt and the body, the locking feature, or both together.

The following exemplary embodiments further describe optional aspects of the presently disclosed technology and are part of this Detailed Description. These exemplary embodiments are set forth in a format substantially akin to claims (each with numerical designations followed by a capital letter), although they are not technically claims of the present application. The following exemplary embodiments refer to each other in dependent relationships as “embodiments” instead of “claims.”

1A. A combination comprising:
a device for administering a drug; and
secondary packaging configured to surround the device, the secondary packaging being configured to resist or prevent inadvertent opening thereof, the secondary packaging comprising a body defining a cavity for at least partially surrounding the device for administering the drug, wherein, when the device is within the cavity.

2A. The combination embodiment 1A, wherein device is a nasal drug delivery device.

3A. The combination according to embodiment 2A, wherein the nasal drug delivery device is reconfigurable between a non-activated configuration wherein the nasal drug delivery device does not release or administer the drug and an activated configuration wherein the nasal drug delivery device does release or administer the drug.

1B. Secondary packaging configured to prevent inadvertent actuation of an inhaler, the secondary packaging comprising a body defining a cavity for at least partially surrounding the inhaler, the body including means for preventing movement of the inhaler that would otherwise release or administer drug from the inhaler.

1C. Secondary packaging configured to hold a device for administering a drug, the secondary packaging comprising:
a body defining a cavity for at least partially surrounding the device for administering the drug; and
a cap movable with respect to the body between a closed position and an open position,
wherein at least one of the body and the lid includes an active polymer component formed of a base polymer entrained with 3 A molecular sieve.

2C. The secondary packaging of embodiment 1C, wherein the body has a base and a sidewall extending upwardly therefrom.

3C. The secondary packaging of embodiment 2C, wherein an end of the sidewall opposite the base including a rim that defines an opening leading to the cavity.

4C. The secondary packaging of embodiment 3C, wherein the cap is pivotally attached to the body between the closed position in which the cap and the body form a moisture-tight seal and an open position in which the opening of the body is exposed.

5C. The secondary packaging of embodiment 4C, wherein the cap includes an annular skirt extending downwardly from a periphery of a base of the cap, the skirt having a first end proximate the base and an opposing second free end.

6C. The secondary packaging of embodiment 4C or 5C, wherein the cap includes a tamper evident feature therein or thereon.

7C. The secondary packaging of embodiment 4C or 5C, wherein the cap includes a latch therein or thereon to maintain the cap in the closed position with respect to the body.

8C. The secondary packaging of embodiment 3C, wherein the rim extends laterally outwardly from the sidewall and parallel to the base of the body.

9C. The secondary packaging of any one of embodiments 1C-8C, wherein an interior surface of the base of the cap includes a stepped configured having two spaced-apart surfaces that each extend parallel to the base of the body when the cap is in the closed position.

10C. The secondary packaging of embodiment 9C, wherein at least one of the two spaced-apart surfaces is configured to contact at least a portion of the device to prevent the device from being reconfigured from the non-activated position to an activated position.

11C. The secondary packaging of any one of embodiments 1C-10C, wherein the base of the cap is planar or arcuate.

1D. Secondary packaging configured to hold a device for administering a drug, the secondary packaging comprising:
a body defining a cavity for at least partially surrounding the device for administering the drug; and
a cap movable with respect to the body between a closed position and an open position, the cap including a latch therein or thereon to maintain the cap in the closed position with respect to the body,
wherein at least one of the body and the lid includes an active polymer component formed of a base polymer entrained with 3 A molecular sieve, and
wherein at least one of the body and the cap are formed of a two-shot injection molding process.

2D. The secondary packaging of embodiment 1D, wherein the body and cap combine to form a vial.

1E. A combination comprising:
a nasal medicament delivery system; and
secondary packaging configured to hold the nasal medicament delivery system, the secondary packaging being in the form of a vial comprising:
a body defining a cavity for at least partially surrounding the device for administering the drug; and
a cap movable with respect to the body between a closed position and an open position, the cap including a latch therein or thereon to maintain the cap in the closed position with respect to the body,
wherein at least one of the body and the lid includes an active polymer component formed of a base polymer entrained with 3 A molecular sieve, and
wherein at least one of the body and the cap are formed of a two-shot injection molding process.

1F. A method of making secondary packaging configured to hold a device for administering a drug, the method comprising:
Two-shot injection molding a body and a cap of the secondary packaging,
wherein at least one of the body and the cap includes an active polymer component formed of a base polymer entrained with 3 A molecular sieve.

1G. Secondary packaging configured to hold a device for administering a drug, the secondary packaging comprising:
a body defining a cavity for at least partially surrounding the device for administering the drug;
a cap attached to at least a portion of the body by a hinge, the cap being movable with respect to the body between a closed position and an open position, the body and cap together defining an internal compartment for housing the device when the cap is in the closed position; and
3 A molecular sieve located within the internal compartment.

2G. The secondary packaging of embodiment 1G, wherein the 3 A molecular sieve is part of an active polymer component, the active polymer component further including a base polymer and optionally a channeling agent.

1H. Secondary packaging configured to hold a device for administering a drug, the secondary packaging comprising:
a body defining a cavity for at least partially surrounding the device for administering the drug; and
a cap attached to at least a portion of the body by a hinge, the cap being movable with respect to the body between a closed position and an open position,
wherein at least one of the body and the lid includes or is attached to an active polymer component comprising 3 A molecular sieve.

2H. The secondary packaging of embodiment 1H, wherein after 7 days of the cap being in the closed position the 3 A molecular sieve does not create additional pressure within the body.

3H. The secondary packaging of embodiment 1H, wherein after 7 days of the cap being in the closed position the 3 A molecular sieve produces less off gassing than 4 A molecular sieve, such that the 3 A molecular sieve does not create additional pressure within the body when the cap is in the closed position.

4H. The secondary packaging of any one of embodiment 1H-3H, wherein the body and the cap are placed in an environment of at least 40° Celsius.

5H. The secondary packaging of any one of embodiments 1H-3H, wherein the body and the cap are placed in an environment of at least 50° Celsius.

6H. The secondary packaging of embodiment 1H, wherein a pressure within the body when the cap is in the closed position decreases after 7 days.

7H. The secondary packaging of embodiment 6H, wherein the pressure within the body when the cap is in the closed position is less than 50 mBars after 6 days.

8H. The secondary packaging of any one of embodiment 1H-7H, wherein the body has a base and a sidewall extending upwardly therefrom.

9H. The secondary packaging of embodiment 8H, wherein an end of the sidewall opposite the base includes a rim that defines an opening leading to the cavity.

10H. The secondary packaging of embodiment 9H, wherein the cap includes an annular skirt extending downwardly from a periphery of a base of the cap, the skirt having a first end proximate the base and an opposing second free end.

11H. The secondary packaging of embodiment 9H or 10H, wherein the cap includes a tamper evident feature therein or thereon.

12H. The secondary packaging of embodiment 9H or 10H, wherein the cap includes a latch therein or thereon to maintain the cap in the closed position with respect to the body.

13H. The secondary packaging of embodiment 9H, wherein the rim extends laterally outwardly from the sidewall and parallel to the base of the body.

14H. The secondary packaging of any one of embodiment 1H-13H, wherein an interior surface of the base of the cap includes a stepped configured having two spaced-apart surfaces that each extend parallel to the base of the body when the cap is in the closed position.

15H. The secondary packaging of embodiment 14H, wherein at least one of the two spaced-apart surfaces is configured to contact at least a portion of the device to prevent the device from being reconfigured from the non-activated position to an activated position.

16H. The secondary packaging of any one of embodiment 1H-15H, wherein the base of the cap is planar or arcuate.

17H. The secondary packaging of any one of embodiment 1H-16H, wherein the entrained polymer composition comprises a base polymer and optionally a channeling agent.

18H. The secondary packaging of any one of embodiment 1H-17H, wherein at least one of the body and the cap are formed of a two-shot injection molding process.

19H. The secondary packaging of any one of embodiment 1H-18H, wherein the body and cap combine to form a vial.

1I. A combination comprising:
a nasal medicament delivery system; and
secondary packaging configured to hold the nasal medicament delivery system, the secondary packaging being in the form of a vial comprising:
   a body defining a cavity for at least partially surrounding the device for administering the drug; and
   a cap movable with respect to the body between a closed position and an open position, the cap including a latch therein or thereon to maintain the cap in the closed position with respect to the body,
   wherein at least one of the body and the lid includes an active polymer component comprising 3 A molecular sieve, and
   wherein at least one of the body and the cap are formed of a two-shot injection molding process.

1J. A method of making secondary packaging configured to hold a device for administering a drug, the method comprising:
two-shot injection molding a body and a cap of the secondary packaging,
inserting an active polymer component comprising 3 A molecular sieve into one of the body and the cap.

1K. A method of preventing a container from inadvertently opening due to a pressure buildup within the container, optionally when the container is located in an environment having a temperature of 50 degrees Celsius or more, the method comprising:
providing the container with an active polymer agent comprising 3 A molecular sieve.

2K. The method of embodiment 1K, further comprising:
maintaining a cap of a body of the container in a closed position that renders the container moisture tight for at least 7 days,
wherein a pressure within the head space of the container decreases after the 7 days, optionally decreasing below 100 mBars.

While the presently disclosed technology has been described in detail and with reference to specific examples thereof, it will be apparent to one skilled in the art that various changes and modifications can be made therein without departing from the spirit and scope thereof. It is understood, therefore, that the presently disclosed technology is not limited to the particular embodiments disclosed, but it is intended to cover modifications within the spirit and scope of the present presently disclosed technology.

What is claimed is:

1. Secondary packaging configured to hold a device for administering a drug, the secondary packaging comprising:
a body defining a cavity for at least partially surrounding the device for administering the drug;
a cap attached to at least a portion of the body by a hinge, the cap being movable with respect to the body between a closed position and an open position, the body and cap together defining an internal compartment for housing the device when the cap is in the closed position; and
an active polymer component located within the internal compartment, the active polymer component being configured to receive at least a portion of the device therein, the active polymer component comprising a mixture of 3 A molecular sieve and a base polymer.

2. The secondary packaging of claim 1, where the body is cylindrical and comprises a base and a sidewall extending upwardly therefrom.

3. The secondary packaging of claim 2, wherein an end of the sidewall opposite the base includes a rim that defines an opening leading to the cavity.

4. The secondary packaging of claim 3, wherein the cap has an annular skirt extending downwardly from a periphery of a base of the cap, the skirt having a first end proximate the base and an opposing second free end.

5. The secondary packaging of claim 4, wherein the rim has an outer surface comprising an undercut relative to a central axis of the body, the undercut having a surface that mates with a corresponding surface of the skirt in a snap fit closure configuration when the cap is closed, wherein the snap fit closure configuration inhibits inadvertent opening of the cap and forms a moisture-tight seal between the body and the cap.

6. The secondary packaging of claim 2, wherein the cap further comprises a latch that engages mating geometry of the cap in the closed position to retain the cap in the closed position and thereby inhibit inadvertent opening of the cap.

7. The secondary packaging of claim 6, wherein the latch comprises at least one opening and the body includes at least on rib configured to mate with the at least one opening in a snap-fit configuration.

8. The secondary packaging of claim 6, further comprising a plug or a lip seal depending from a base of the cap, the plug or lip seal being configured to engage an inner surface of the sidewall to form a moisture tight seal between the cap and the body.

9. The secondary packaging of claim 5, further comprising an elastomeric seal on an underside of the cap configured to engage at least a portion of the body when the cap is in the closed position to form a moisture tight seal between the cap and the body.

10. The secondary packaging of claim 2, wherein the active polymer component is attached to or integral with the sidewall of the body, the active polymer component comprising a central axial cavity configured to receive and surround a portion of the device.

11. The secondary packaging of claim 10, the central cavity terminating at an annular ledge configured to provide a seating surface for an annular flange of a body of the device.

12. The secondary packaging of claim 1, wherein when the cap is in the closed position, the secondary packaging is moisture tight and wherein at a storage temperature of 50° C., the active polymer component generates an initial release of gas causing pressure to build up within the internal compartment and to reach a peak pressure, followed by a decrease of pressure within the internal compartment while the cap remains in the closed position.

13. The secondary packaging of claim 12, wherein the peak pressure is reached within one day after the cap is placed in the closed position.

14. The secondary packaging of any one of claim 12, wherein the peak pressure is from 80 mBars to 120 mBars and the final pressure is from 40 mBars to 60 mBars.

15. The secondary packaging of claim 12, wherein pressure built up within the internal compartment is not sufficient to cause the cap to inadvertently open.

16. The secondary packaging of claim 1, wherein the cap comprises a base, at least a portion of which is vaulted to project vertically above the rim when the cap is in the closed position and thus form a recess within the cap configured to house a portion of the device.

17. A medication delivery system comprising the secondary packaging of claim 1 and a drug delivery device stored therein,
wherein the drug delivery device comprises a body having a discharge end defining a drug discharge port and an opposed actuator end, the drug delivery device including a drug reservoir having a drug stored therein, the drug delivery device further including an actuator for ejecting the drug from the reservoir through the discharge port, the actuator extending out of the actuator end of the body, the actuator being linearly movable from an extended position to a depressed position, the discharge port being sealed when the actuator is in the extended position and the discharge port being open when the actuator is in the depressed position, the actuator being operable to discharge the drug from the reservoir upon axial movement of the actuator from the extended position to the depressed position,
wherein the drug delivery device is configured for intranasal administration and the drug, when administered, is in the form of a liquid, an aerosol, a gel or a powder, and
wherein the drug is a formulation comprising one or more of the following active pharmaceutical ingredients: Naloxone, Ketamine, Midazolam, Fentanyl, Lorazepam, Nalmefene, Epinephrine, Apomorphine, Cetrorelix, Loxapine, Eletriptan, Ketorolac, Glucagon and Olanzapine.

18. A method of providing shelf stability to a drug contained in a drug delivery device that is stored within secondary packaging, the secondary packaging having a cap and body, the cap being pivotable with respect to the body about a hinge, shelf stability is maintained by providing an environment with low relative humidity or no relative humidity and by ensuring that that cap does not inadvertently open while the drug delivery device is stored within the secondary packaging, the method comprising:
locating an active polymer component within an internal compartment formed by the cap and the body when the cap is in a closed position with respect to the body, the active polymer component comprising a base polymer and 3 A molecular sieve.

19. The method of claim 18, further comprising:
inserting the drug delivery device into the secondary packaging; and
moving the cap from an open position to a closed position with respect to the body, thereby enclosing the drug delivery device within the secondary packaging,
wherein the secondary packaging prevents inadvertent actuation of the drug delivery device and creates a low relative humidity or no relative humidity environment in a closed cavity of the secondary packaging.

20. The method of claim 19, further comprising:
moving the cap from the closed position with respect to the body;
removing the drug delivery device from the secondary packaging; and
administering the drug within the drug delivery device to a patient.
